(19) United States
(12) Patent Application Publication
(10) Pub. No.: US 2026/0166293 A1
(43) Pub. Date: Jun. 18, 2026

(54) System and Method to Detect and Disrupt Occlusion

(71) Applicant: Bard Perioheral Vascular, Inc., Franklin Lakes, NJ (US)

(72) Inventors: Marvin Clyde Culla Vergara, Gilbert, AZ (US); William E. Parmentier, Gilbert, AZ (US)

(21) Appl. No.: 19/127,040

(22) PCT Filed: Nov. 4, 2022
(86) PCT No.: PCT/US2022/048969

§ 371 Date: May 2, 2025

(51) Int. Cl. A61M 39/02; A61B 17/00; A61B 17/12; A61M 25/06; A61M 25/10

(52) U.S. Cl. CPC A61M 39/0208; A61B 17/0057; A61B 17/12109; A61M 25/0606; A61M 25/1002

(57) ABSTRACT

An occlusion detection and disruption system includes a vascular access device (VAD) including a sensor configured to detect an attribute, e.g. fluid pressure. The VAD can be communicatively coupled with an external console configured to determine a threshold value for the attribute and determine any deviation from the threshold value that indicates the onset or presence of an occlusion. When the system detects an occlusion, e.g. thrombosis, biofilm, etc., the system can automatically activate a disruptor system to dislodge or ablate the occlusion. The disruptor system can include a piezoelectric actuator and/or an electro-magnetic actuator coupled with the VAD and powered by a battery and/or induction coil. The system can be communicatively coupled with other VAD's to aggregate additional information and improve the accuracy of detecting the onset and formation of occlusions.

### BACKGROUND

Vascular access devices (VAD) such as catheters, ports, and the like, which include a portion disposed subcutaneously or intravascularly, can be susceptible to a buildup of biofilms which in turn can cause thrombosis to form in, on, or around a portion of the VAD. Such thromboses can reduce the patency of the VAD or occlude the VAD entirely. Current standards of care include flushing the VAD on a regular basis in order to dislodge any such occlusions. However, such flushing's may not fully clear the occlusion. Further, the clinician must estimate how often to perform the flushing events with little information to guide the decision. If the interval between flushing events is too frequent this can cause undue discomfort to the patient. If the interval between flushing events is not frequent enough the occlusion may become established and occlude the VAD entirely, preventing further flushing events and resulting in replacement of the VAD altogether. What is needed therefore, is a system configured to automatically detect the presence of an occlusion and activate a disruptor system included as part of the VAD. The disruptor system is configured to dislodge or ablate the occlusion, restoring patency to the VAD. This can clear an occlusion before the occlusion becomes fully established and blocks the VAD entirely, mitigating premature replacement of the VAD.

In addition, little is known about the processes surrounding the onset and formation of thrombi etc., which can occlude the VAD. Such information about the onset and formation of such occlusions can help develop improved standards of care. Accordingly, what is also needed is a system configured to detect and record one or more attributes, e.g. fluid attributes, patient attributes, etc. Information about these attributes along with the occurrence of an occlusion event can be analyzed to determine which attributes can be used as indicators of an occlusion, or the onset of an occlusion.

### SUMMARY

Briefly summarized, embodiments disclosed herein are directed to a system and method for determining the onset and presence of occlusions, e.g. thrombosis, etc., within a vascular access device (VAD), and automatically activating a disruptor system to dislodge or ablate the cause of the occlusion and restore patency to the VAD. Embodiments include a system and method for detecting changes in one or more attributes to determine the onset of the formation of biofilms, thrombi, or similar causes of occlusion. The system can record these attributes, along with the occurrence(s) of occlusions to determine which attributes, or combinations thereof, are indicators of the onset of occlusions. Once the onset, or presence of an occlusion is determined, the system can automatically initiate a disruptor system to dislodge or ablate the occlusion and restore patency to the VAD. Such systems can provide greater insight as to the causes or early warning signs behind the onset of thrombi or similar occlusions. Further, such systems can automatically trigger disruptor systems included as part of the VAD that can disrupt the thrombi before the occlusion becomes established.

Disclosed herein is a subcutaneous vascular access system including, a vascular access device defining a fluid pathway, and including a sensor configured to detect one or more attributes including a fluid pressure within the fluid pathway, and a disruptor system, and a console configured to communicate with the vascular access device, the console including one or more processors and a non-transitory computer-readable medium having stored thereon logic, when executed by the one or more processors, causes operations including, determining a change in the attribute relative to a threshold to determine the presence of an occlusion, and activating the disruptor system to dislodge the occlusion and restore patency to the fluid pathway.

In some embodiments, the disruptor system includes a piezoelectric actuator communicatively coupled with the console and, when activated, configured to provide physical pressure waves to dislodge or ablate the occlusion.

In some embodiments, the disruptor system includes an electro-magnetic actuator communicatively coupled with the console and, when activated, configured to provide electromagnetic radiation to dislodge or ablate the occlusion.

In some embodiments, the one or more attributes further includes a fluid attribute or a patient attribute, the fluid attribute includes one or more of an electrical conductance/impedance, viscosity, flow rate, color, and opacity, the patient attribute includes one or more of body temperature, heart rate, ECG, and breathing rate.

In some embodiments, the vascular access device includes a subcutaneous port and catheter extending therefrom, the distal tip of the catheter is configured to be disposed within a vasculature of a patient.

In some embodiments, the port includes a port body defining a reservoir and is in fluid communication with a lumen of the catheter, the sensor is disposed within one of the reservoir or the lumen of the catheter.

In some embodiments, the vascular access device further includes a communications logic, and one of a battery, rechargeable battery, or an induction coil, configured to power the sensor or the communications logic.

In some embodiments, the disruptor system includes one or more actuators disposed on an outer surface of the vascular access device, an inner surface of the fluid pathway, or within a wall structure of the vascular access device.

In some embodiments, the threshold is predetermined and stored by the console.

In some embodiments, the threshold is dynamically determined by the console from two or more attribute measurements from the sensor.

In some embodiments, the console is configured to receive information from one of a network, an external computing device or a second vascular access device to determine the threshold.

Also disclosed is a method of restoring patency to a vascular access device including, detecting one or more attributes by a sensor, the sensor disposed in a fluid pathway of a vascular access device and configured to detect at least a fluid pressure attribute, determining a threshold value for the one or more attributes, determining if the one or more attributes is greater or lesser than the threshold value to determine the presence of an occlusion, and activating a disruptor system to dislodge or ablate the occlusion and restore patency to the fluid pathway of the vascular access device.

In some embodiments, activating the disruptor system includes activating a piezoelectric actuator coupled with the vascular access device to provide physical pressure waves to dislodge or ablate the occlusion.

In some embodiments, activating the disruptor system includes activating an electromagnetic actuator coupled with the vascular access device to provide electro-magnetic radiation to dislodge or ablate the occlusion.

In some embodiments, the one or more attributes further includes a fluid attribute or a patient attribute, the fluid attribute includes one or more of an electrical conductance/impedance, viscosity, flow rate, color, and opacity, the patient attribute includes one or more of body temperature, heart rate, ECG, and breathing rate.

In some embodiments, the vascular access device includes a subcutaneous port and catheter extending therefrom, the distal tip of the catheter is configured to be disposed within a vasculature of a patient.

In some embodiments, the port includes a port body defining a reservoir and is in fluid communication with a lumen of the catheter, the sensor is disposed within one of the reservoir or the lumen of the catheter.

In some embodiments, the method further includes wirelessly communicating information between the vascular access device and a console disposed externally, the vascular access device further including a communications logic, and one of a battery, rechargeable battery, or an induction coil, configured to power the sensor and the communications logic.

In some embodiments, the disruptor system includes one or more actuators disposed on an outer surface of the vascular access device, an inner surface of the fluid pathway, or within a wall structure of the vascular access device.

In some embodiments, determining a threshold value includes retrieving a predetermined threshold.

In some embodiments, determining a threshold value includes deriving a dynamic threshold from two or more attribute measurements from the sensor over time.

In some embodiments, determining a threshold value includes retrieving information from one of a network, an external computing device or a second vascular access device.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 shows a schematic view of an exemplary occlusion detection and disruptor system, in accordance with embodiments disclosed herein.

FIG. 2 shows an exploded view of a vascular access device of the system of FIG. 1, in accordance with embodiments disclosed herein.

FIGS. 3A-3B show schematic views of a vascular access device and exemplary patterns of attributes that indicate the onset or presence of an occlusion, in accordance with embodiments disclosed herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

### Terminology

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Singular forms of “a,” “an,” and “the” include plural references unless the context clearly dictates otherwise.

In the following description, certain terminology is used to describe aspects of the invention. For example, in certain situations, the term “logic” is representative of hardware, firmware or software that is configured to perform one or more functions. As hardware, logic may include circuitry having data processing or storage functionality. Examples of such circuitry may include, but are not limited or restricted to a hardware processor (e.g., microprocessor with one or more processor cores, a digital signal processor, a programmable gate array, a microcontroller, an application specific integrated circuit “ASIC,” etc.), a semiconductor memory, or combinatorial elements.

Alternatively, logic may be software, such as executable code in the form of an executable application, an Application Programming Interface (API), a subroutine, a function, a procedure, an applet, a servlet, a routine, source code, object code, a shared library/dynamic load library, or one or more instructions. The software may be stored in any type of a suitable non-transitory storage medium, or transitory storage medium (e.g., electrical, optical, acoustical or other form of propagated signals such as carrier waves, infrared signals, or digital signals). Examples of non-transitory storage medium may include, but are not limited or restricted to a programmable circuit; semiconductor memory; non-persistent storage such as volatile memory (e.g., any type of random access memory “RAM”); or persistent storage such as non-volatile memory (e.g., read-only memory “ROM,” power-backed RAM, flash memory, phase-change memory, etc.), a solid-state drive, hard disk drive, an optical disc drive, or a portable memory device. As firmware, the executable code may be stored in persistent storage. In an embodiment, the logic described herein may rely on heuristics, machine learning, artificial intelligence (A.I.), neural networks, or other data processing techniques or similar artificial intelligence schema to perform the described functionality.

The term “computing device” should be construed as electronics with the data processing capability and/or a capability of connecting to any type of network, such as a public network (e.g., Internet), a private network (e.g., a wireless data telecommunication network, a local area network “LAN”, etc.), or a combination of networks. Examples of a computing device may include, but are not limited or restricted to, the following: a server, an endpoint device (e.g., a laptop, a smartphone, a tablet, a “wearable” device such as a smart watch, augmented or virtual reality viewer, or the like, a desktop computer, a netbook, a medical device, or any general-purpose or special-purpose, user-controlled electronic device), a mainframe, internet server, a router; or the like.

A “message” generally refers to information transmitted in one or more electrical signals that collectively represent electrically stored data in a prescribed format. Each message may be in the form of one or more packets, frames, HTTP-based transmissions, or any other series of bits having the prescribed format.

As used herein “wireless” communication modalities can include but not limited to Wifi, Bluetooth, Near Field Communications (NFC), cellular Global System for Mobile Communication (“GSM”), electromagnetic (EM), radio frequency (RF), combinations thereof, or the like.

The term “computerized” generally represents that any corresponding operations are conducted by hardware in combination with software and/or firmware.

Labels such as “left,” “right,” “upper”, “lower,” “top,” ,“bottom,” “front,” “back,” and the like are used for convenience and are not intended to imply, for example, any particular fixed location, orientation, or direction. Instead, such labels are used to reflect, for example, relative location, orientation, or directions.

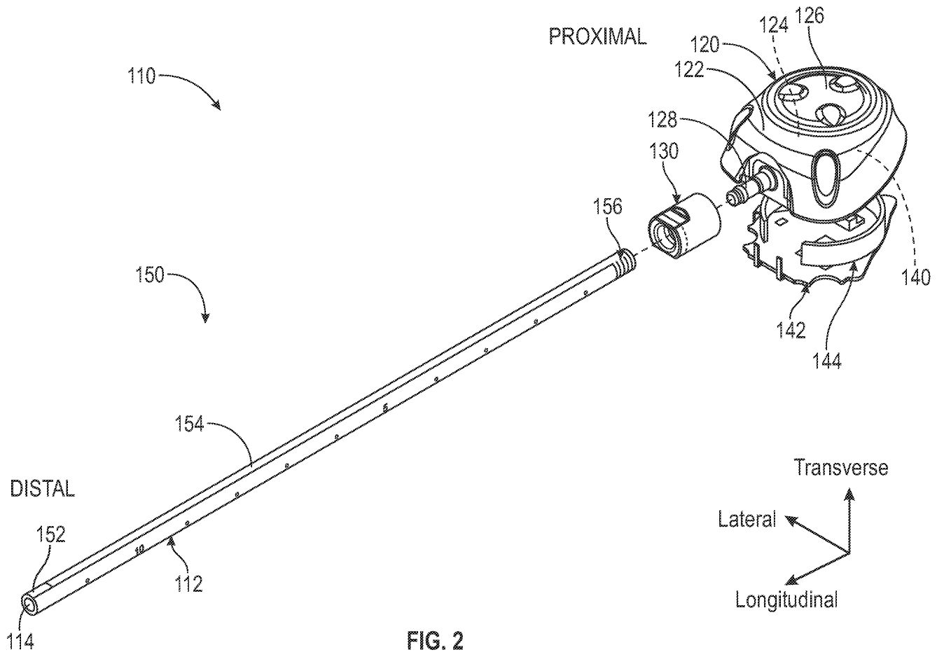

To assist in the description of embodiments described herein, as shown in FIG. 2, a longitudinal axis extends substantially parallel to an axis of the catheter. A lateral axis extends normal to the longitudinal axis, and a transverse axis extends normal to both the longitudinal and lateral axes.

With respect to “proximal,” a “proximal portion” or a “proximal end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal end portion, or the proximal length of the catheter can include the proximal end of the catheter; however, the proximal portion, the proximal end portion, or the proximal length of the catheter need not include the proximal end of the catheter. That is, unless context suggests otherwise, the proximal portion, the proximal end portion, or the proximal length of the catheter is not a terminal portion or terminal length of the catheter.

With respect to “distal,” a “distal portion” or a “distal end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal end portion, or the distal length of the catheter can include the distal end of the catheter; however, the distal portion, the distal end portion, or the distal length of the catheter need not include the distal end of the catheter. That is, unless context suggests otherwise, the distal portion, the distal end portion, or the distal length of the catheter is not a terminal portion or terminal length of the catheter.

Unless defined otherwise, all technical and scientific terms used herein have the same meaning as commonly understood by those of ordinary skill in the art.

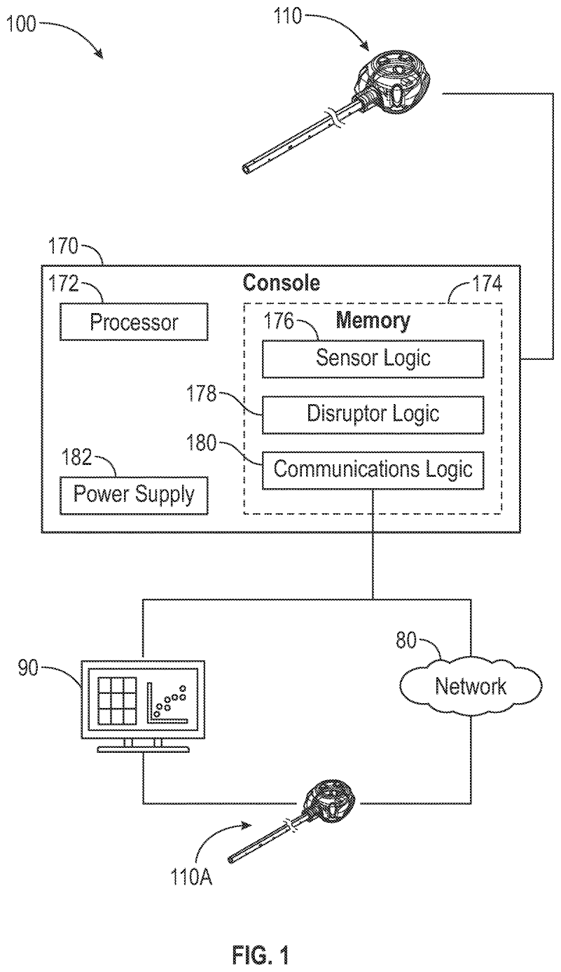

FIG. 1 shows an occlusion detection and disruption system (“system”) 100 generally including a vascular access device (VAD) 110 and a console 170 communicatively coupled thereto. At least a portion of the VAD 110 can be disposed internally to a patient and the console 170 can be disposed externally to the patient. Exemplary VADs 110 can include single lumen catheters, multi-lumen catheters, midline catheters, central vascular catheters (CVC), peripherally inserted central catheters (PICC), dialysis catheters, split-tip catheters, subcutaneous ports, combinations thereof, or the like.

In an embodiment, the console 170 can be a standalone device, smart phone, mobile device, or similar computing device. The console 170 can include one or more of a processor 172, non-transitory memory 174, one or logic engines such as a sensor logic 176, disruptor logic 178, and communications logic 180, and a power supply 182. One or more of the sensor logic 176, disruptor logic 178, communications logic 180 can be disposed on the circuitry 142 of the VAD 110, the console 170, or both.

In an embodiment, the sensor logic 176 can be communicatively coupled with a sensor 140 disposed on the VAD 110 and can be configured to detect a change in an attribute and determine the onset or presence of an occlusion in or on the VAD 110, as described in more detail herein. In an embodiment, the disruptor logic 178 can be communicatively coupled with a disruptor system 150 coupled with the VAD 110 and can be configured to actuate the disruptor system 150 to dislodge or ablate an occlusion, e.g. thrombosis, in or on the VAD.

In an embodiment, the console 170 can be communicatively coupled with a network 80, one or more additional computing device(s) 90, and/or one or more additional VAD's 110A. Exemplary computing devices 90 can include mobile devices, laptops, computers, mainframes, servers, electronic health record (EHR) servers, or the like. The console 170 can retrieve additional information from the network 80, computing device 90, and/or additional VAD's 110A to further improve the accuracy in determining the onset of an occlusion, as described in more detail herein.

FIG. 2 shows further details of an exemplary VAD 110 generally including a catheter tube 112 and a port 120. The catheter tube 112 includes an elongate body defining one or more catheter lumens 114. A distal end of the catheter tube 112 can be configured to be disposed within a vasculature of a patient. To note, a distal portion of the catheter tube 112 can include one or more openings, fluid flow structures, valves, split-tip structures, or the like.

In an embodiment, a proximal end of the catheter tube 112 can be in fluid communication with a subcutaneous access port (“port”) 120. However, it will be appreciated that a proximal end of the catheter tube 112 can be in fluid communication with one or more catheter hubs, extension legs, valves, or the like, and can be disposed subcutaneously, or can be disposed externally on a skin surface and can be in place of, or in addition to, the port 120, or combinations thereof. In an embodiment, the proximal end of the catheter tube 112 can be coupled to the port using a cathlock device (“cathlock”) 130.

In an embodiment, the port 120 can generally include a port body 122 defining a reservoir 124. The port 120 can include a needle penetrable septum 126 disposed over the reservoir 124 and can provide percutaneous access thereto by an access needle. The port 120 can include a port stem 128 that is in fluid communication with the reservoir 124. A proximal end of the catheter tube 112 can be coupled to the port stem 128 and optionally secured in place using the cathlock 130.

In an embodiment, the VAD 110 can further include a sensor 140 configured to detect a attribute, such as a fluid attribute and/or a patient attribute. Exemplary fluid attributes can include one or more of fluid pressure, electrical conductance/impedance, viscosity, flow rate, color, opacity, or the like. Exemplary patient attributes can include one or more of body temperature, heart rate, blood pressure, ECG, breathing rate, or the like. To note, as used herein, the sensor 140 can include a single sensor device configured to detect one or more attributes, or an array of sensors each configured to detect one or more attributes.

As an illustrative example, the sensor 140 can be a pressure sensor configured to detect a pressure of a fluid disposed within the VAD 110, i.e. within one or more of the reservoir 124, a lumen of the port stem 128, and a lumen 114 of the catheter tube 112. In an embodiment, the sensor 140 can be disposed on a wall of the reservoir, an inner wall of the lumen of the port stem 128, an inner wall of the catheter lumen 114, or combinations thereof. The system 100 can detect a change, e.g. increase, in fluid pressure relative to a threshold value and can determine the presence of an occlusion. The system 100 can then actuate the disruptor system 150 to dislodge or ablate the occlusion and restore patency, as described in more detail herein.

As another illustrative example, the sensor 140 can be configured to detect the electrical conductivity, or changes thereof, in the fluid (e.g. blood) passing over an outer surface of the VAD or through a lumen of the VAD. A drop in conductivity relative to a threshold value would indicate that a thrombosis is forming. The system 100 can then determine when a change in conductivity is significant enough relative to a threshold to trigger the disruptor system 150 and dislodge or ablate the occlusion (thrombosis). Advantageously, the system 100 can automatically detect and disrupt any occlusion without having to rely on periodic flushing's by a clinician which may also be overlooked or performed too infrequently to maintain patency to the VAD.

In an embodiment, the VAD 110 can include circuitry 142 (e.g. circuit board, processor(s), non-transitory memory, logic, combinations thereof, or the like), configured to receive information from the sensor 140 and communicate the information, either wired or wirelessly, with the console 170. Exemplary wireless communication modalities can include WiFi, bluetooth, Near Field Communication (NFC), radio frequency (RF), induction, or the like. In an embodiment, the VAD 110 can include a power source 144, for example, one or more of a battery, rechargeable battery, induction coil, or the like, configured to power one or more of the sensor 140, circuitry 142, and a disruptor system 150.

In an embodiment, the VAD 110 can include a disruptor system 150 configured to dislodge or disrupt (ablate) any occlusion disposed on or in the VAD 110. As used herein, the disruptor system 150 can use one or more modalities to disrupt the occlusion, including but not limited to, physical (mechanical) pressure wave energy, acoustic energy, ultrasonic energy, electromagnetic (EM) energy, radio frequency (RF) energy, thermal energy, resistive heating, combinations thereof, or the like. By way of a non-limiting example, the VAD 110 can include one or more piezo actuator(s) (“actuator”) 152 configured to receive electrical energy and produce physical (mechanical) pressure waves. The VAD 110 can include an electrical conduit 154 configured to electrically couple the actuator 152 with one or both of the circuitry 142 and power source 144.

In an embodiment, actuator 152 can be disposed on the catheter tube 112 and the electrical conduit 154 can extend longitudinally along the catheter tube 114 to an electrical coupling 156 disposed at a proximal end of the catheter tube 112. The electrical coupling 156 can be configured to provide electrical communication between the electrical conduit 154 and actuator 152 disposed on the catheter tube 112 and the circuitry 142 and/or power source 144 disposed on the port 120. In an embodiment, the cathlock 130 can further include an electrical coupling 156, as described herein.

In an embodiment, the one or more actuator(s) 152 can be disposed on an outer surface of the catheter tube 112, on an inner surface of the catheter lumen 114, within a wall of the catheter tube 112, on an outer surface of the port 120, an inner surface of the port 120 (e.g. inner surface of the reservoir 124, or the lumen of the port stem 128), or combinations thereof. In an embodiment, the actuator(s) 152 can be disposed at one or more longitudinal positions along the catheter tube 112. In an embodiment, the actuator(s) 152 can be disposed at one or more radial positions about the catheter tube 112.

In an embodiment, the actuator 152 can produce energy waves at a given frequency, or at a range of frequencies, a given amplitude, or at a range of amplitudes, or combinations thereof. In an embodiment, the electrical signals sent from the circuitry 142 and/or power source 144 can modify the frequency/amplitude of waves produced by the one or more actuators 152. In an embodiment, different actuators 152 can be configured to produce different frequencies, amplitudes, or ranges thereof, or vary the frequencies/amplitudes over time.

In an embodiment, the energy produced by the actuator 152 can disrupt, dislodge, or ablate an occlusion, such as a biofilm, thrombosis, or the like, that is disposed on a surface of the VAD 110. In an embodiment, the disruptor system 150 can use one or more modalities to disrupt the occlusion, as described herein. Advantageously, two or more modalities can work in unison to provide a synergistic effect on improving the disruption of the occlusion.

In an exemplary method of use, the sensor logic 176 can receive information from the sensor 140 about an attribute, e.g. fluid attribute and/or patient attribute. For example, the sensor 140 can detect a change in fluid pressure, electrical conductivity of the fluid, heart rate, breathing rate, combinations thereof, or the like, and determine the onset, or the presence, of an occlusion. The disruptor logic 178 can then receive information from the sensor logic 176 that an occlusion is present, or is starting to form, and can actuate the disruptor system 150 autonomously to dislodge or ablate the source of the occlusion.

In an embodiment, one or more of the sensor logic 176 and the disruptor logic 178 can communicate with a network 80 and/or an external computing device 90 to further analyze the information, determine the onset, or the presence, of an occlusion, and/or actuate the disruptor system 150. In an embodiment, the system 100 can determine a change in attribute relative to a predetermined threshold. The predetermined threshold can be stored locally on one or both of the VAD 110 or the console 170, or can be retrieved from the network 80 and/or additional computing device 90. Advantageously, the system 100 can automatically detect an occlusion and actuate the disruptor system 150 to dislodge occlusion. This prevents the occlusion becoming established in between flushing's and mitigates relying on a clinician to flush the VAD in a timely manner.

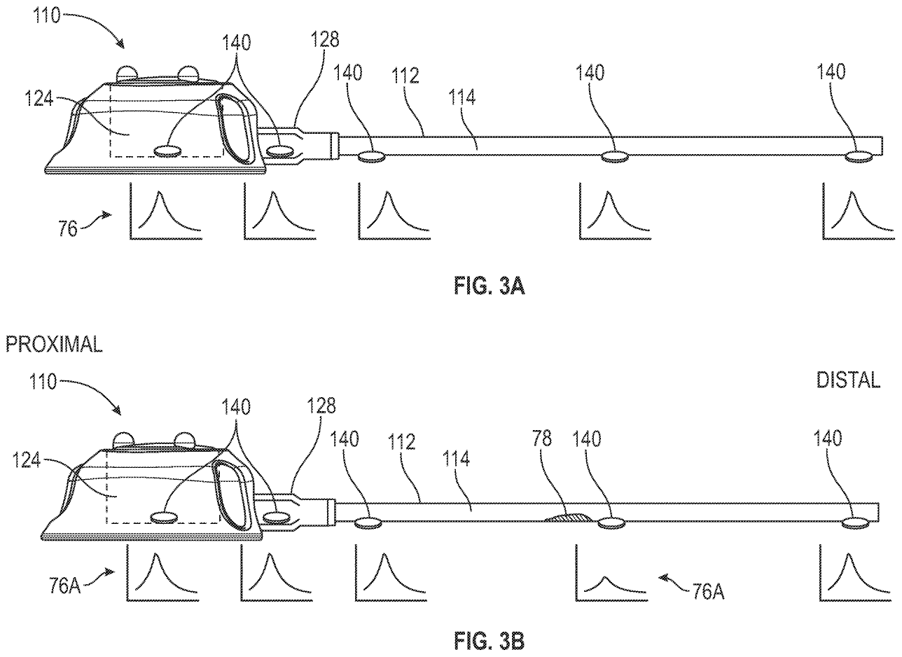

In an embodiment, the system 100 can determine a dynamic threshold level for one or more attributes. The sensor logic 176 can receive information from the sensor(s) 140 and determine a baseline attribute value, e.g. a baseline pressure level. To note, the baseline attribute value can also vary over time. For example, as shown in FIGS. 3A-3B, when the port 120 is accessed by an access needle, a bolus of fluid can be introduced through the access needle, into the reservoir 124, through the port stem 128, through the catheter lumen 114 and into the vasculature. One or more sensors 140 disposed at one or more positions along the fluid pathway can detect an increase in fluid pressure, followed by decrease in pressure as the fluid bolus passes through the fluid pathway. The sensor logic 176 can detect distinctive patterns 76 of pressure changes along the fluid pathway and establish a base line pressure change for fluid pressure within the VAD 110 before, during, and after a fluid bolus is introduced. Over repeated fluid boli, the system 100 can increase the accuracy and determine a “normal” base line pressure that is distinctive to the specific VAD system 110, VAD placement, and patient (FIG. 3A).

Advantageously, this “dynamic” threshold can be determined by the system 100 and accurately reflect the specifics of the VAD system 110. For example, each configuration of VAD 110 can vary depending on the catheter type, material, number of lumens, cross-sectional shape of lumen, catheter tube length, port type, catheter tip flow structures, etc. Further, the location of catheter placement, or difference between patients can also affect the baseline attribute profile. Further, the accuracy of the dynamic threshold can be improved over time with increased data gathered from the VAD system 110, and/or from data gathered from one or more additional VAD systems 110A disposed in other patients and communicated to the system 100 by way of the external computing device 90 and/or network 80.

In an embodiment, as shown in FIG. 3B, once the sensor logic 176 has established the dynamic base line specific to the VAD system 110, the sensor logic 176 can then determine any deviations 76A in the pattern of pressure changes across the fluid pathway. These deviations in attribute values can be analyzed by the sensor logic 176 to determine if they are caused by an occlusion 78, or are an indication of the onset of an occlusion 78.

In an embodiment, once an occlusion has been detected, the disruptor system 178 can retrieve such information from the sensor logic 176 and determine when to actuate the disruptor system 150. Further the disruptor system 150 can determine when to activate the disruptor system 150, when to deactivate the disruptor system 150, how long to actuate the disruptor system 150, the amount or changes in amplitude or frequency or changes thereof of wave energy, the modalities of wave energy employed (physical/mechanical wave energy, electrical wave energy), or combinations thereof, in order to dislodge the occlusion and restore patency to the VAD.

In an embodiment, once the disruptor system 150 has completed an actuation cycle, the disruptor system 150 can query the sensor logic 176 to determine if the occlusion is cleared sufficiently, i.e. a return to a “normal” base line pressure pattern. If not, the disruptor system 150 can repeat the disruption cycle, or modify the disruption cycle providing differing modalities of wave energy, amplitude, frequencies, combinations thereof, or the like.

In an embodiment, one or more of the sensor(s) 140 can be configured to detect an attribute of the patient. Exemplary attributes of the patient can include, but not limited to, body temperature, heart rate, ECG, blood pressure, electrical conductivity of the blood, or the like. Advantageously, the sensor logic 176 can include patient attribute information from sensors 140 when established a dynamic base line. Similarly, any deviations in patient attributes from the dynamic base line (e.g. increase in body temperature, increase/decrease in blood pressure), may indicate an onset of occlusion formation. Further, the system 100 can determine which combinations of changes in fluid or patient attributes that may indicate the onset of occlusion formation.

For example, the system 100 can gather data from the VAD 110, and/or from VAD's 110A placed in other patients, and determine indicators for the onset of occlusions. As a simplified example, the patients'body temperature may increase prior to the formation of an occlusion as determined by a change in fluid pressure within the fluid pathway. As such the system can learn from the data gathered and determine one or more indicators from either patient attributes and/or VAD attributes that indicates the onset of an occlusion and can trigger the disruptor system 150 as appropriate. Advantageously, the system 100 can determine attributes that indicate the early onset of occlusions. The system 100 can then use these newly identified indicator attributes to trigger the disruptor 150 system and disrupt or dislodge the occlusion before becoming established and inhibiting flushing the VAD altogether.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A subcutaneous vascular access system, comprising:
a vascular access device defining a fluid pathway, and including a sensor configured to detect one or more attributes including a fluid pressure within the fluid pathway, and a disruptor system; and
a console configured to communicate with the vascular access device, the console including one or more processors and a non-transitory computer-readable medium having stored thereon logic, when executed by the one or more processors, causes operations including:
determining a change in the attribute relative to a threshold to determine the presence of an occlusion; and
activating the disruptor system to dislodge the occlusion and restore patency to the fluid pathway.

2. The subcutaneous vascular access system according to claim 1, wherein the disruptor system includes a piezoelectric actuator communicatively coupled with the console and, when activated, configured to provide physical pressure waves to dislodge or ablate the occlusion.

3. The subcutaneous vascular access system according to claim 1, wherein the disruptor system includes an electro-magnetic actuator communicatively coupled with the console and, when activated, configured to provide electromagnetic radiation to dislodge or ablate the occlusion.

4. The subcutaneous vascular access system according to claim 1, wherein the one or more attributes further includes a fluid attribute or a patient attribute, the fluid attribute includes one or more of an electrical conductance/impedance, viscosity, flow rate, color, and opacity, the patient attribute includes one or more of body temperature, heart rate, ECG, and breathing rate.

5. The subcutaneous vascular access system according to claim 1, wherein the vascular access device includes a subcutaneous port and catheter extending therefrom, the distal tip of the catheter is configured to be disposed within a vasculature of a patient.

6. The subcutaneous vascular access system according to claim 5, wherein the port includes a port body defining a reservoir and is in fluid communication with a lumen of the catheter, the sensor is disposed within one of the reservoir or the lumen of the catheter.

7. The subcutaneous vascular access system according to claim 1, wherein the vascular access device further includes a communications logic, and one of a battery, rechargeable battery, or an induction coil, configured to power the sensor or the communications logic.

8. The subcutaneous vascular access system according to claim 1, wherein the disruptor system includes one or more actuators disposed on an outer surface of the vascular access device, an inner surface of the fluid pathway, or within a wall structure of the vascular access device.

9. The subcutaneous vascular access system according to claim 1, wherein the threshold is predetermined and stored by the console.

10. The subcutaneous vascular access system according to claim 1, wherein the threshold is dynamically determined by the console from two or more attribute measurements from the sensor.

11. The subcutaneous vascular access system according to claim 10, wherein the console is configured to receive information from one of a network, an external computing device or a second vascular access device to determine the threshold.

12. A method of restoring patency to a vascular access device, comprising:
detecting one or more attributes by a sensor, the sensor disposed in a fluid pathway of a vascular access device and configured to detect at least a fluid pressure attribute;
determining a threshold value for the one or more attributes;
determining if the one or more attributes is greater or lesser than the threshold value to determine the presence of an occlusion; and
activating a disruptor system to dislodge or ablate the occlusion and restore patency to the fluid pathway of the vascular access device.

13. The method according to claim 12, wherein activating the disruptor system includes activating a piezoelectric actuator coupled with the vascular access device to provide physical pressure waves to dislodge or ablate the occlusion.

14. The method according to claim 12, wherein activating the disruptor system includes activating an electromagnetic actuator coupled with the vascular access device to provide electro-magnetic radiation to dislodge or ablate the occlusion.

15. The method according to claim 12, wherein the one or more attributes further includes a fluid attribute or a patient attribute, the fluid attribute includes one or more of an electrical conductance/impedance, viscosity, flow rate, color, and opacity, the patient attribute includes one or more of body temperature, heart rate, ECG, and breathing rate.

16. The method according to claim 12, wherein the vascular access device includes a subcutaneous port and catheter extending therefrom, the distal tip of the catheter is configured to be disposed within a vasculature of a patient.

17. The method according to claim 16, wherein the port includes a port body defining a reservoir and is in fluid communication with a lumen of the catheter, the sensor is disposed within one of the reservoir or the lumen of the catheter.

18. The method according to claim 12, further including wirelessly communicating information between the vascular access device and a console disposed externally, the vascular access device further including a communications logic, and one of a battery, rechargeable battery, or an induction coil, configured to power the sensor and the communications logic.

19. The method according to claim 12, wherein the disruptor system includes one or more actuators disposed on an outer surface of the vascular access device, an inner surface of the fluid pathway, or within a wall structure of the vascular access device.

20. The method according to claim 12, wherein determining a threshold value includes retrieving a predetermined threshold.

21. The method according to claim 12, wherein determining a threshold value includes deriving a dynamic threshold from two or more attribute measurements from the sensor over time.

22. The method according to claim 12, wherein determining a threshold value includes retrieving information from one of a network, an external computing device or a second vascular access device.
